(19) United States
(12) Patent Application Publication
(10) Pub. No.: US 2023/0292994 A1
(43) Pub. Date: Sep. 21, 2023

(54) ENDOSCOPE ARRANGEMENT

(71) Applicant: Schölly Fiberoptic GmbH, Denzlingen (DE)

(72) Inventors: Daniel HÄRTER, Emmendingen (DE); Alexander KÖHLER, Freiburg (DE); Stefan SCHRÖER, Freiburg (DE); Lutz LABUSCH, Emmendingen (DE)

(73) Assignee: Schölly Fiberoptic GmbH, Denzlingen (DE)

(21) Appl. No.: 18/014,777

(22) PCT Filed: Jul. 14, 2021
(86) PCT No.: PCT/EP2021/069603

§ 371 Date: Jan. 6, 2023

(30) Foreign Application Priority Data
Jul. 21, 2020 (DE) 102020119172.3

(51) Int. Cl. A61B 1/00; A61B 1/05; A61B 1/045

(52) U.S. Cl. CPC A61B 1/00117; A61B 1/05; A61B 1/00006; A61B 1/045; A61B 1/00114

(57) ABSTRACT

An endoscope arrangement (1) including a camera head (2) with at least one distal image sensor (4) and an image-processing unit (3) which is connected to the camera head (2) via a connecting line (14). The connecting line (14) has at least one optical line (15) for transmitting image information. The connecting line (14) additionally has an electrical line (16) for serially transmitting electrical signals, it being possible to transmit electrical control signals bidirectionally via this electrical line.

### TECHNICAL FIELD

The invention relates to an endoscope arrangement having a camera head with at least one distally arranged image sensor and an image processing unit (CCU) which is connected to the camera head via a connecting line.

### BACKGROUND

In such endoscope arrangements, it is known to transmit the image information as a video data stream via the connecting line from the image sensor to the CCU. Here, it is common to transmit the video data via electrical or optical lines.

### SUMMARY

The object of the invention is to simplify the apparatus structure in connection with an optical transmission.

This object is achieved according to the invention by an endoscope arrangement of the aforementioned type with one or more of the features described herein.

According to the invention, the endoscope arrangement is characterized in that the connecting line comprises an electrical line for serial transmission of signals.

In this way, there is no need for converters in the camera head that extract signals mixed in with the optical video data stream, for example control signals. Thus, a return channel in the optical line of the unidirectional video data stream can also be dispensed with, which significantly reduces the apparatus complexity, especially since such converters usually have to be programmed in a complex way.

Another advantage is that there is preferably only one electrical line in the connecting line via which the signals are transmitted.

In one embodiment, at least one clock signal and a first control signal, which are fed to the image sensor in the camera head, are transmitted via the electrical line. This way, no processing or other conditioning of the control signals for the image sensor is necessary in the camera head. In particular, the control signals are fed directly to the image sensor, i.e. without an interposed processor. An advantage is a very simple construction of the camera head without a processor. In particular, this can also reduce heat generation in the camera head, so that the potential heat input into the environment can also be reduced.

In one embodiment, a second control signal is transmitted via the electrical line and is fed to additional functional units in the camera head, in particular to memory, sensors and/or actuating elements. In this way, additional functional units can be connected to the image processing unit without adversely affecting the control of the image sensor and without the need for a processor in the camera head.

In one embodiment, the electrical line is designed as a two-wire line, in particular as a twisted pair, shielded twisted pair (STP) or coaxial cable. In this way, serial transmission of the signals is possible with little effort. Serial transmission protocols, such as I2C, are standardized and easy to implement with specific interface modules, especially without a processor.

In one embodiment, the image processing unit has a converter which combines the signals for serial transmission via the electrical line. The camera head has a converter which separates the serially transmitted signals again. In this way, different signals, such as control signals or clock signals, can be transmitted via one line without influencing each other.

In one embodiment, the converter(s) is/are each designed as a Gigabit Multimedia Serial Link (GMSL) serializer/deserializer. In this way, a large amount of data can be transmitted via a simple two-wire line, and even over greater distances of up to 15 meters with a shielded line.

Another advantage is that such converters, in particular also GMSL serializers/deserializers, are available as individual components that do not require any additional hardware, in particular no processor and no specific programming. The technical complexity is therefore very low, which makes the endoscope arrangement easy and inexpensive to manufacture. Since the converters work bidirectionally, a bidirectional transmission of the signals is possible without any problems.

In one embodiment, the image processing unit has a main processor which feeds the signals separately to the converter. In this way, the main processor can generate a plurality of separate signals intended for different functional units in the camera head. These are separated by the converter in the camera head and fed directly to the functional units. A processor in the camera head is therefore no longer necessary, which also eliminates the need for complex programming. The serial signal transmission is therefore completely transparent, as if the main processor were directly connected to the functional unit in the camera head via separate lines in each case.

In an alternative embodiment, the image processing unit has a main processor which is connected via the electrical line, in particular directly, to a functional unit, in particular a memory, of the camera head.

In one embodiment, the main processor is connected to a coprocessor of the camera head via an electrical line of the connecting line. An operating program and control signals are transmitted from the main processor to the coprocessor via this electrical line.

The electrical connection between the main processor and the coprocessor can, for example, be made via the electrical line or via a separate, second electrical line.

The coprocessor can be designed, for example, for image processing and control of the image sensor, in particular as an FPGA. In this way, the coprocessor can receive the programming of the FPGA from the main processor, for example. The functionality of the coprocessor can thus be customizable.

In one embodiment, the connecting line has an optical conductor for each optical image channel. In this way, several videos can be transmitted.

In one embodiment, serializers/deserializers are connected upstream and downstream of the optical conductor to transmit the optical image channels on the optical conductor present. In this way it is possible, for example, to transmit a plurality of image channels on one optical conductor.

In one embodiment, an optical conductor is associated with a dedicated optical fiber in the connecting line. In this way, a good separation of the image channels and a high bandwidth for transmission is possible.

Such a fiber can be a single-mode or multimode fiber. Especially in multimode fibers, an optical conductor can also be assigned to one mode of a fiber.

In a further embodiment, connection by means of a plurality of connecting lines is conceivable. Each connecting line contains one or more optical lines and an electrical line in this case.

### BRIEF DESCRIPTION OF THE DRAWINGS

The invention is explained in more detail below by means of exemplary embodiments with reference to the accompanying drawings.

The figures show:

FIG. 1: a first example of an endoscope arrangement according to the invention, and

FIG. 2: a second example of an endoscope arrangement according to the invention.

### DETAILED DESCRIPTION

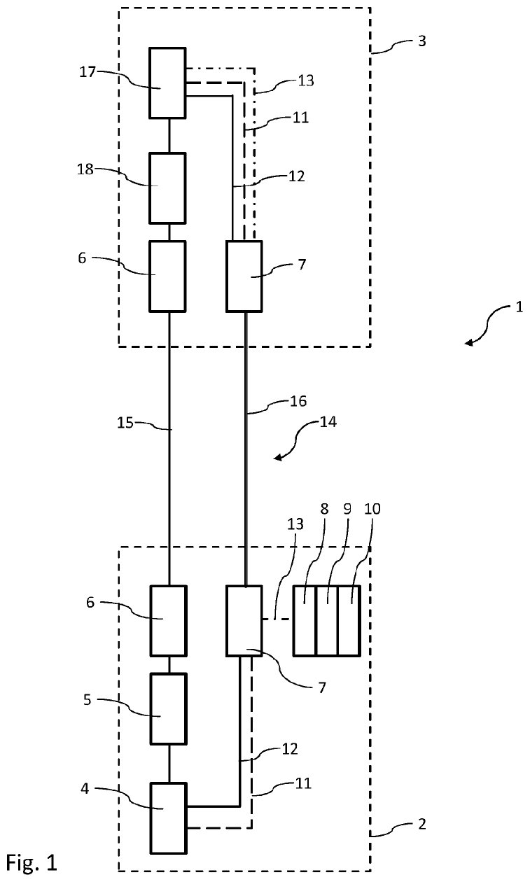

The endoscope arrangement 1 of FIG. 1 has a camera head 2 and an image processing unit 3, which are connected to each other via a connecting line 14. The connecting line 14 has an optical line 15 and an electrical line 16. The optical line 15 serves to transmit image information from the camera head 2 to the image processing unit 3. The optical line 15 has at least one fiber, which may be designed as a single-mode or multimode fiber. Preferably, the optical line 15 has as many fibers or modes as there are image channels.

The electrical line 16 is a two-wire line, which can be designed as a twisted pair, shielded twisted pair (STP) or coaxial cable.

The camera head 2 has an image sensor 4 connected to a serializer 5 for image data. The serializer 5 reads the image data from the image sensor 4 and prepares the image data for transmission. The serializer 5 is a standard component that can be used without further programming. The serialized image data are converted in an optical transducer 6 into optical signals, which are coupled into the optical line 15.

The camera head 2 also has a converter 7 for electrical signals. In the example, this converter 7 is a Gigabit Multimedia Serial Link (GMSL) serializer/deserializer, which is available as a standard module and can also be used without further programming. The converter 7 is connected to the electrical conductor 16 of the connecting line 14. From the converter 7, a clock signal 11 and a first control signal 12 lead directly to the image sensor 4.

Through the converter 7, i.e. via the GMSL connection of the electrical conductor 16, a voltage supply to the electrical components of the camera head 2 can also take place.

The camera head 2 is designed without a processor or any other component that requires programming.

A second control signal 13 leads from the converter 7 to further functional units, such as a memory 8, a sensor 9 and/or an operating element 10. Naturally, there can be other, different or even fewer elements in the functional unit. There can also be a plurality of functional units present, which are connected to the converter via further separate control signals. The number of electrical signals that can be routed separately by the converter is only limited by the module used.

The first control signal 12 and the second control signal 13 are preferably designed as serial signals with a two-wire line, in particular as an I2C bus.

The image processing unit 3 has a main processor 17 which outputs the first control signal 12, the second control signal 13 and the clock signal 11 as separate signals. These three signals are connected to a converter 7 of the image processing unit 3 via individual electrical lines. The converter 7 corresponds to the converter 7 of the camera head 2. Accordingly, the converter 7 combines the separate signals for serial transmission via the electrical line 16 of the connecting line 14. An advantage is that different signals are transmitted serially via only one electrical line without influencing each other. In this case, the signals can be transmitted bidirectionally so that, for example, inputs from the operating elements 10 and measured values from the sensors 9 can be transmitted to the main processor.

The image processing unit 3 further has an optical transducer 6 which receives the optical signals from the optical line and converts them into electrical signals. The electrical image data are fed to a deserializer 18. From the deserializer 18, the image data are fed to the main processor 17 for further image processing. The main processor 17 is designed as an FPGA, for example. An advantage is that the image data can be transmitted unidirectionally and there is no need to provide a return channel for control signals in the optical line. In this way, the serializers 5 and deserializers 18 can be designed to be much simpler, in particular as standard modules that can be used without programming.

In one example, the converter 7 and the serializer 5 and the converter 7 and the deserializer 18 are in each case implemented in one module.

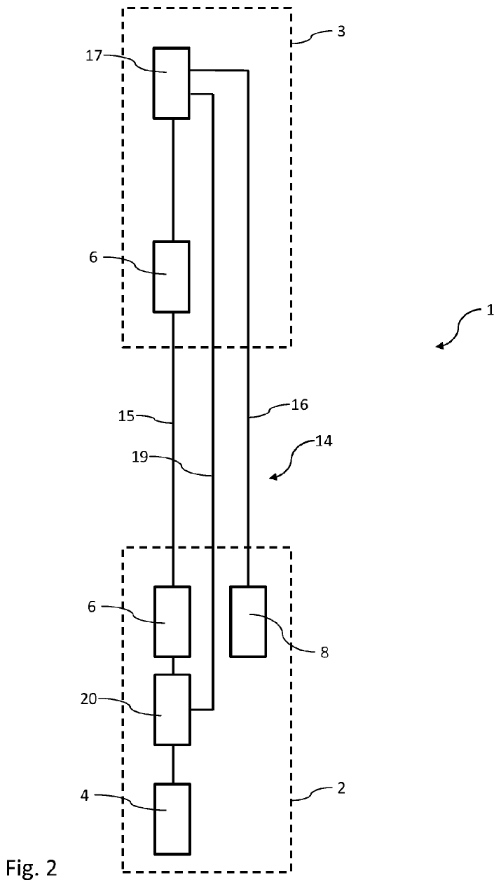

FIG. 2 shows an alternative example of an endoscope arrangement 1 with a camera head 2 and an image processing unit 3, which are connected to each other via a connecting line 14. The connecting line 14 here has a second electrical line 19 in addition to the optical line 15 and the electrical line 16.

The electrical line 16 is designed as a two-wire line for serial data transmission and connects the main processor 17 directly to a memory 8 of the camera head 2. In addition to the memory 8, other or further functional units of the camera head 2 may also be connected.

In this example, there is no serializer 5 for image data in the camera head 2 and no deserializer 18 in the image processing unit 3. The camera head 2 has a coprocessor 20 arranged instead between the image sensor 4 and the optical transducer 6. In this example, the image data can be transmitted via a corresponding number of fibers and/or modes of the optical line 15. However, there may also be a plurality of optical lines 15.

However, the coprocessor 20 can also perform serialization and/or simple image processing in the camera head 2 directly. In the example, the optical line 15 is designed as a CRS/SDI connection.

The main processor 17 and the coprocessor 20 are directly connected via the second electrical line 19. Control commands can be sent to the coprocessor via this second electrical line 19, for example to control the image sensor 4. However, the operating program or a boot program can also be transmitted from the main processor 17 to the coprocessor 20. In this way, for example, the FPGA of the coprocessor 20 can be configured. It is also possible to make changes to the coprocessor 20 thereby, for example to change the image processing in the coprocessor.

### LIST OF REFERENCE SIGNS

1 Endoscope arrangement
2 Camera head
3 Image processing unit
4 Image sensor
Serializer
6 Optical transducer
7 Converter
8 Memory
9 Sensor
10 Operating element
11 Clock signal
12 First control signal
13 Second control signal
14 Connecting line
15 Optical line
16 Electrical line
17 Main processor
18 Deserializer
19 Second electrical line
20 Coprocessor

1. An endoscope arrangement (1), comprising:
a camera head (2) with at least one distally arranged image sensor (4);
an image processing unit (3) connected to the camera head (2) via a connecting line (14), the connecting line (14) having: a) at least one optical line (15) for transmitting image information, and b) at least one electrical line (16) for serial transmission of signals.

2. The endoscope arrangement (1) as claimed in claim 1, wherein the electrical line (16) is configured to transmit at least one clock signal (11) and a first control signal (12), which are fed to the image sensor (4) in the camera head (2).

3. The endoscope arrangement (1) as claimed in claim 2, wherein the electrical line (16) is configured to transmit a second control signal (13) that is fed to additional functional units in the camera head (2), including at least one of a memory (8), sensors (9), or actuating elements (10).

4. The endoscope arrangement (1) as claimed in claim 1, wherein the electrical line (16) comprises a two-wire.

5. The endoscope arrangement (1) as claimed in claim 1, wherein the image processing unit (3) has a converter (7) which combines the signals (11, 12, 13) for serial transmission via the electrical line (16), and the camera head (2) has a camera head converter (7) which separates the signals (11, 12, 13) transmitted serially via the electrical line (16).

6. The endoscope arrangement (1) as claimed in claim 5, wherein the image processing unit (3) has a main processor (17) which feeds the signals (11, 12, 13) separately to the converter (7).

7. The endoscope arrangement (1) as claimed in claim 1, wherein the image processing unit (3) has a main processor (17) which is connected via the electrical line (16) to a functional unit of the camera head (2).

8. The endoscope arrangement (1) as claimed in claim 7, wherein the main processor (17) is connected to a coprocessor (20) of the camera head (2) via an electrical line (19) of the connecting line (14) that is configured to transmit an operating program and control from the main processor (17) to the coprocessor (20).

9. The endoscope arrangement (1) as claimed in claim 1, wherein the connecting line (14) has an optical conductor (15) for each optical image channel.

10. The endoscope arrangement (1) as claimed in claim 1, further comprising an optical conductor (15) associated with at least one of a dedicated optical fiber in the connecting line or a mode of a fiber.

11. The endoscope arrangement (1) as claimed in claim 4, wherein the two-wire line is a twisted pair, shielded twisted pair or coaxial cable.

12. The endoscope arrangement (1) as claimed in claim 5, wherein the converter and the camera head converter are GMSL converters.

13. The endoscope arrangement (1) as claimed in claim 7, wherein the functional unit is a memory.

14. The endoscope arrangement (1) as claimed in claim 9, further comprising a serializer (5) and deserializer (18) connected upstream and downstream of the optical conductor (15), respectively, configured to transmit the optical image channels on the optical conductor (15).
